(19) United States
(12) Patent Application Publication
(10) Pub. No.: US 2021/0179332 A1
(43) Pub. Date: Jun. 17, 2021

(54) DEVICE FOR PACKAGING AND APPLICATION OF A LIQUID OR SEMI-SOLID MATERIAL

(71) Applicant: The Tapemark Company, West St. Paul, MN (US)

(72) Inventors: Cormac H. Lyons, Inver Grove Heights, MN (US); Steven A. Rau, Farmington, MN (US); Julie A. Cardin, Hastings, MN (US)

(21) Appl. No.: 17/124,167

(22) Filed: Dec. 16, 2020

Related U.S. Application Data
(60) Provisional application No. 62/949,292, filed on Dec. 17, 2019.

(51) Int. Cl. B65D 75/58; B65D 75/56; B65D 75/32

(52) U.S. Cl. CPC B65D 75/5855; B65D 75/326; B65D 75/56

(57) ABSTRACT

Various embodiments provide a package for containing and applying a substance. The package can include a handle layer, a carrier layer, an applicator, and an enclosing layer. The handle layer can include: a first handle portion; a second handle portion; and a joining portion. The carrier layer can include: a first surface; and a second surface. The joining portion of the handle layer can be connected to the first surface of the carrier layer. The applicator can contain the substance. The first handle portion and the second handle portion are configured to move from a first state to a second state, wherein, in the first state, the first handle portion and the second handle portion are parallel with the carrier layer, and, in the second state, the first handle portion and the second handle portion are not parallel to the carrier layer.

This application claims the benefit of U.S. Provisional Application No. 62/949,292, filed Dec. 17, 2019, the content of which is herein incorporated by reference in its entirety.

### FIELD

Embodiments herein relate to a package for containing and applying a substance.

### BACKGROUND

Many different types of packages are made with the intent to contain a product, such as a liquid or cream. Once the package is opened, the product needs to be applied to a desired location. Some packages are designed to contain the product and to dispense the product when a tear or hole is made in the package and the package is compressed. Other packages are designed to be opened thereby exposing the product. A brush, pad, finger, or other object can be inserted into the package and into the product to remove at least a portion of the product. In some scenarios the user does not want to touch the product with his/her hands or an object in order to apply the product.

### SUMMARY

Various embodiments provide a package for containing and applying a substance, the package can include a handle layer that can include a first handle portion, a second handle portion, and a joining portion between the first handle portion and the second handle portion, a carrier layer is included having a first surface, and a second surface opposite from the first surface, wherein the joining portion of the handle layer is connected to the first surface of the carrier layer, an applicator containing the substance, and an enclosing layer coupled to the second surface of the carrier layer, wherein the enclosing layer, and the carrier layer define a cavity with the applicator disposed in the cavity, such that the applicator is disposed between the carrier layer and the enclosing layer, wherein the first handle portion and the second handle portion are configured to move from a first state to a second state, wherein, in the first state, the first handle portion and the second handle portion are parallel with the carrier layer, and, in the second state, the first handle portion and the second handle portion are not parallel to the carrier layer.

In an embodiment, the applicator holds the substance in pores of the applicator.

In an embodiment, the applicator includes an absorbent material, cloth, paper, porous polymer, foam, or sponge.

In an embodiment, the applicator is bonded to the second surface of the carrier layer.

In an embodiment, a first score defines a boundary between the first handle portion and the joining portion, and a second score defines a boundary between the second handle portion and the joining portion.

In an embodiment, the first score and the second score extend at least 10% through the handle layer and not more than 90% through the handle layer.

In an embodiment, the first handle portion and the second handle portion rotate in opposite directions to move between the first state to the second state.

In an embodiment, in the first state, the first handle portion is coplanar with the second handle portion.

In an embodiment, the first handle portion and the second handle portion each include a gripping element.

In an embodiment, the gripping element includes at least one of an aperture, a projection, and a recess.

In an embodiment, the handle layer includes stiff paper, thermoplastic material, polyester, or polypropylene.

In an embodiment, a first angle between the first handle portion in the first state and the first handle portion in the second state is at least 15° and not more than 130°, and a second angle between the second handle portion in the first state and the second handle portion in the second state is at least 15° and not more than 130°.

In an embodiment, the carrier layer includes a foil polymer paper laminate.

In an embodiment, a portion of the enclosing layer is removeable.

In an embodiment, a portion of the enclosing layer is fixed to the package, and a portion of the enclosing layer is not fixed to the package.

In an embodiment, micro-perforations define a boundary between the portion of the enclosing layer that is removeable and a portion of the enclosing layer which is not removeable.

In an embodiment, the enclosing layer is coupled to the carrier layer with a peelable seal.

Various embodiments provide a package for containing and applying a substance, the package is included having a handle layer is included having a first handle portion can include a first end and a second end, a second handle portion can include a first end and a second end, and a joining portion between the first handle portion and the second handle portion, wherein the first end of the first handle portion is adjacent to the joining portion and the first end of the second handle portion is adjacent to the joining portion, a carrier layer is included having a first surface, and a second surface opposite from the first surface, wherein the joining portion of the handle layer is connected to the first surface of the carrier layer, an applicator containing the substance, and an enclosing layer coupled to the carrier layer, wherein the enclosing layer, and the carrier layer define a cavity with the applicator disposed in the cavity, such that the applicator is disposed between the carrier layer and the enclosing layer, wherein the first handle portion and the second handle portion are configured to move from a first state to a second state, wherein in the first state the second end of the first handle portion and the second end the second handle portion are further apart than when the first handle portion and the second handle portion are in the second state.

Various embodiments provide a method of making a packing, the method can include providing a handle layer web, a carrier layer web, and an enclosing layer web, coupling applicators to the carrier layer web, placing a substance on the applicators, scoring the handle layer web to divide handle portions from joining portions, forming a combined web by coupling the enclosing layer web to the carrier layer web to enclose the applicators and coupling the handle layer web to the carrier layer web, and cutting at least one individual package around at least a portion of an outer package perimeter to separate the individual package from the combined web, wherein a first handle portion and a second handle portion of the individual package are configured to move from a first state to a second state, wherein, in the first state, the first handle portion and the second handle portion are parallel with the carrier layer, and, in the second state, the first handle portion and the second handle portion are not parallel to the carrier layer.

In an embodiment, cutting the individual package includes die cutting or rotary cutting.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a perspective view of a package in accordance with various embodiments herein.

FIG. 2 is an exploded view of the package shown in FIG. 1 in accordance with various embodiments herein.

FIG. 3 is a top view of the package shown in FIG. 1 in accordance with various embodiments herein.

FIG. 4 is a side view of the package shown in FIG. 1 in accordance with various embodiments herein.

FIG. 5 is an end view of the package shown in FIG. 1 in accordance with various embodiments herein.

FIG. 6 is a bottom view of the package shown in FIG. 1 in accordance with various embodiments herein.

FIG. 7 is a perspective view of a package in accordance with various embodiments herein.

FIG. 8 is a bottom perspective view of the package shown in FIG. 7 in accordance with various embodiments herein.

FIG. 9 is a top view of the package shown in FIG. 7 in accordance with various embodiments herein.

FIG. 10 is a side view of the package shown in FIG. 7 in accordance with various embodiments herein.

FIG. 11 is a bottom view of the package shown in FIG. 7 in accordance with various embodiments herein.

FIG. 12 is an exploded view of the package shown in FIG. 7 in accordance with various embodiments herein.

FIG. 13 is a perspective view of a handle layer in accordance with various embodiments herein.

FIG. 14 is a side view of the handle layer shown in FIG. 13 in accordance with various embodiments herein.

FIG. 15 is a bottom view of the handle layer shown in FIG. 13 in accordance with various embodiments herein.

FIG. 16 is a perspective view of a carrier layer shown in FIG. 13 in accordance with various embodiments herein.

FIG. 17 is a bottom view of the carrier layer shown in FIG. 16 in accordance with various embodiments herein.

FIG. 18 is a perspective view of the applicator in accordance with various embodiments herein.

FIG. 19 is a side view of the applicator shown in FIG. 18 in accordance with various embodiments herein.

FIG. 20 is a top view of the applicator shown in FIG. 18 in accordance with various embodiments herein.

FIG. 21 is a perspective view of the enclosing layer in accordance with various embodiments herein.

FIG. 22 is a side view of the enclosing layer shown in FIG. 21 in accordance with various embodiments herein.

FIG. 23 is a top view of the enclosing layer shown in FIG. 21 in accordance with various embodiments herein.

FIG. 24 is a side, cross-sectional schematic view of a package in accordance with various embodiments herein.

FIG. 25 is a side, cross-sectional schematic view of the package shown in FIG. 24 in accordance with various embodiments herein.

FIG. 26 is a side, cross-sectional schematic view of the package shown in FIG. 24 in accordance with various embodiments herein.

FIG. 27 is a side, cross-sectional schematic view of the package shown in FIG. 24 in accordance with various embodiments herein.

FIG. 28 is a bottom schematic view of a package in accordance with various embodiments herein.

FIG. 29 is a side, cross-sectional schematic view of a package in accordance with various embodiments herein.

FIG. 30 is a side, cross-sectional schematic view of the package shown in FIG. 29 in accordance with various embodiments herein.

FIG. 31 is a bottom schematic view of a package in accordance with various embodiments herein.

FIG. 32 is a side, cross-sectional schematic view of a package in accordance with various embodiments herein.

FIG. 33 is a side, cross-sectional schematic view of a package in accordance with various embodiments herein.

FIG. 34 is a side, cross-sectional schematic view of a portion of a package in accordance with various embodiments herein.

FIG. 35 is a side, cross-sectional schematic view of the portion of the package shown in FIG. 34 in accordance with various embodiments herein.

FIG. 36 is a perspective view of a package in accordance with various embodiments herein.

FIG. 37 is a perspective view of a package in accordance with various embodiments herein.

FIG. 38 is a flowchart depicting a method in accordance with various embodiments herein.

FIG. 39 is a top view of a portion of various packages in a manufacturing stage in accordance with various embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

Various embodiments of a package are described herein. The package can be configured to contain or hold a substance, such as a substance that is a solid, liquid or partially solid and partially liquid. The package can include a handle portion, such that a user can apply the substance to a desired surface, such as a person's skin, without contacting the substance. In various embodiments, the package can be transitioned from a first state to a second state. In the first state, the handle portion can be planar or flat. In a second state, the handle portion can project away from the remainder of the package, such that the handle portion can be grasped by a user. The user can grasp or hold the handle to apply the substance to a desired surface. An enclosing layer can be at least partially removed from the package to expose the substance. In some embodiments, the enclosing layer is at least partially removed when the package is in the first state, and in other embodiments, the enclosing layer can be at least partially removed when the package is in the second state. The package disclosed herein can allow a user to expose the substance, and apply the substance without directly contacting the substance. The package also provides a structure to use to apply the substance to a receiving surface without using a secondary transfer mechanism, such as a separate brush, pad or application stick.

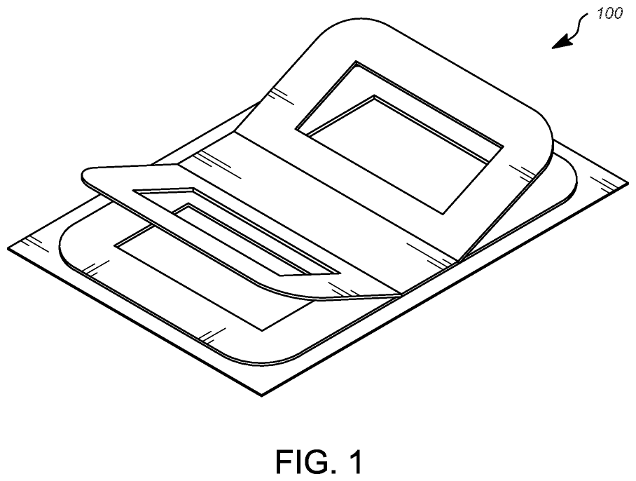

The term “second state” as used herein refers to a position of one or more handle portions that are not planar with a joining portion of the handle layer, not planar with the carrier layer, and can be used by a user to grasp the package. Examples will be described with the understanding that there is a continuum of possible positions in a second state for the handle portions. One example of a second state is shown in FIGS. 1-5, 7-10, and 12-15, where handle portions of the handle layer are rotated by about 25 degrees to extend away from the carrier layer. Another example of a second state is shown in FIGS. 25-27, where handle portions of the handle layers are rotated by about 110 degrees to extend away from the carrier layer and have their ends in contact. Many other examples and ranges of the second state for the handle portions are possible. Similarly, there are a variety of ways that users can grasp the handle portions when they are in the second state, including one-handed options and two-handed options.

Referring now to the figures, FIG. 1 shows a perspective view of a package 100 in accordance with various embodiments herein. The package 100 can be configured to contain a substance between various layers of the package. The package can be configured to apply the substance to a surface, such as a person's skin.

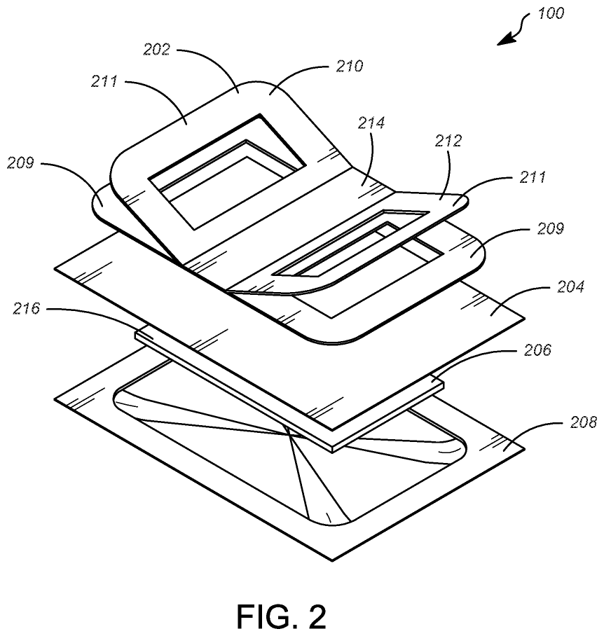

Referring now to FIG. 2, an exploded view of the package 100 shown in FIG. 1 is shown in accordance with various embodiments herein. The package 100 can include a handle layer 202, a carrier layer 204, an applicator 206, an enclosing layer 208, and a substance 216.

The handle layer 202 can include a first handle portion 210 and a second handle portion 212. The handle layer 202 can also include a joining portion 214. The joining portion 214 can extend between the first handle portion 210 and the second handle portion 212, such as to link or join the handle portions.

In various embodiments, the first handle portion 210 and the second handle portion 212 are configured to move from a first state 209 to a second state 211. In various embodiments, the first handle portion 210 and the second handle portion 212 rotate in opposite directions to move between the first state 209 to the second state 211. The handle portions maybe alternatively be in an intermediate state between the first and second states, such as while the handle portions are being moved between the first state and the second state. It should be further understood that the first handle portion 210 and the second handle portion 212 can be in different states at the same time, such as the first handle portion 210 being in the first state 209 and the second handle portion 212 being in the second state 211 as the package 100 is converted to prepare the package 100 to apply the substance.

The FIGS. 1-5, 7-10, and 12-14 herein show two first handle portions 210, one in the first state 209 and one in the second state 211 in the same FIG., to show the positions of the two states relative to each other. However, in reality the package 100 includes only one first handle portion 210. Similarly, these FIGS. show two second handle portions 212, one in the first state 209 and one in the second state 211 in the same FIG., although in reality the package 100 includes only one second handle portion 212.

In various embodiments, the joining portion 214 can be located between the first handle portion 210 and the second handle portion 212. In various embodiments, the joining portion 214 of the handle layer 202 can be connected to the carrier layer 204, such as to a top or first surface of the carrier layer 204. In various embodiments, the joining portion 214 can be connected, joined or coupled to the carrier layer 204 with an adhesive, sealant layer, or a weld, such as a plastic weld.

In various embodiments, the carrier layer 204 and the enclosing layer 208 can define a cavity (described further below) between the carrier layer 204 and the enclosing layer 208. In various embodiments, an applicator 206 can be disposed in the cavity between the carrier layer 204 and the enclosing layer 208. In some embodiments, the cavity can be a sterile environment. In some embodiments, the package 100 can be hermetically sealed.

In various embodiments, the applicator 206 can be connected, joined, bonded or coupled to the carrier layer 204. In some embodiments, the applicator 206 can be bonded to the second surface of the carrier layer 204 via a heat seal, ultrasonic weld, adhesive, interference fit or chemically bonded, such as with a two-part epoxy. The applicator 206 can be connected, joined, bonded or coupled to the second surface of the carrier layer 204, such as opposite from the joining portion 214. In various embodiments, the applicator 206 can contain or hold the substance 216.

In various embodiments, the enclosing layer 208 can be coupled to the second surface of the carrier layer 204, such as on the same side of the carrier layer 204 as the applicator 206. In various embodiments, the enclosing layer 208 can be coupled to the carrier layer 204 with a peelable seal (described further below). In some embodiments, portions of the enclosing layer 208 can be permanently coupled to the carrier layers, such as with an adhesive or a weld. A permanent coupling can be stronger than the layers that are being coupled together, such that the layers themselves would be destroyed prior to the coupling between the two layers being destroyed.

In various embodiment, in the first state 209, the first handle portion 210 and the second handle portion 212 are parallel with the carrier layer 204, and in the second state 211, the first handle portion 210 and the second handle portion 212 are not parallel to the carrier layer 204. In various embodiment, in the first state 209, the first handle portion 210 and the second handle portion 212 are parallel with the each other, and in the second state 211, the first handle portion 210 and the second handle portion 212 are not parallel with each other. In various embodiment, in the first state 209, the first handle portion 210 and the second handle portion 212 are coplanar, and in the second state 211, the first handle portion 210 and the second handle portion 212 are not coplanar.

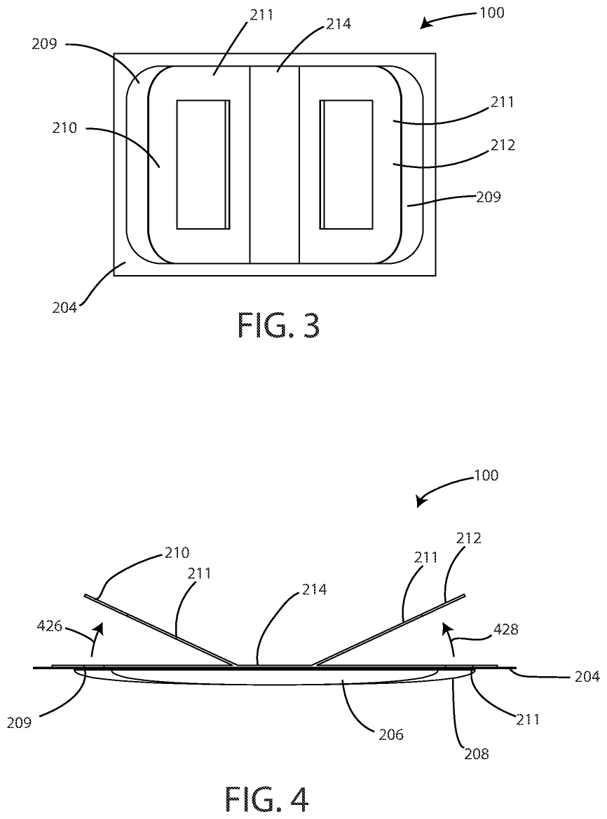

Referring now to FIG. 3, a top view of the package 100 shown in FIG. 1 is shown in accordance with various embodiments herein. FIG. 1 shows the handle layer 202 and the carrier layer 204. The first state 209 and the second state 211 of the first handle portion 210 and the second handle portion 212 are shown. Further, the joining portion 214 is shown joining the first handle portion 210 and the second handle portion 212 together. The joining portion 214 is coupled to the carrier layer 204. In various embodiments, the top or first surface of the carrier layer 204 can have a larger surface area than the top of the handle layer 202 including the first handle portion 210 and the second handle portion, as shown in FIG. 3.

Referring now to FIG. 4, a side view of the package 100 shown in FIG. 1 is shown in accordance with various embodiments herein. Arrow 426 shows the direction of movement of the first handle portion 210 from the first state 209 to the second state 211. Arrow 428 shows the direction of movement of the second handle portion 212 from the first state 209 to the second state 211. In various embodiments, the first handle portion 210 and the second handle portion 212 rotate in opposite directions to move between the first state 209 to the second state 211. From the perspective of the view of FIG. 4, the first handle portion 210 rotates in a clockwise direction and the second handle portion rotates in an opposite counter clockwise direction. In some embodiments, the first handle portion 210 and the second handle portion 212 each rotate around the intersection or connection between the handle portion 210, 212 and the joining portion 214.

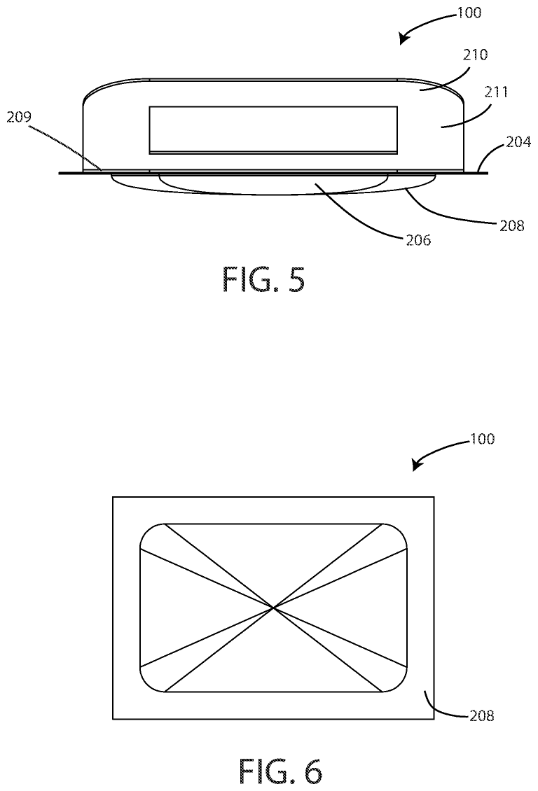

Referring now to FIG. 5, an end view of the package 100 shown in FIG. 1 is shown in accordance with various embodiments herein. As can be seen in FIG. 5, in various embodiments, the first handle portion 210 and the second handle portion 212 in the first state 209 can be conforming, flat, planar, or parallel with the carrier layer 204. In contrast, in the second state 211, the first handle portion 210 and the second handle portion 212 can non-planar and non-parallel compared to each other and/or compared to the carrier layer.

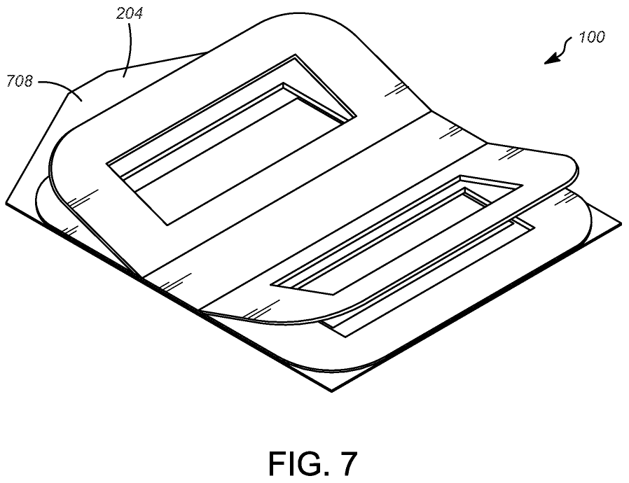

Referring now to FIG. 6, a bottom view of the package 100 shown in FIG. 1 is shown in accordance with various embodiments herein. The package 100 can include an enclosing layer 208. The enclosing layer 208 can be configured to enclose a cavity in which the applicator 206 is disposed within.

FIGS. 7-11 show a package 100 similar to the package shown in FIGS. 1-6. The package 100 shown in FIGS. 7-12 can include an extension. The extension can be held or grasped by a user, such as while removing at least a portion of the enclosing layer to expose the substance 216 or while transitioning the handle portions 210, 212 from the first state 209 to the second state 211.

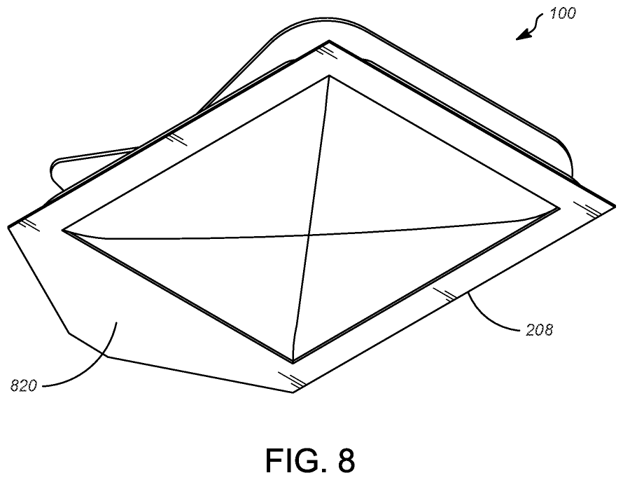

FIG. 7 shows a perspective view of a package 100 is shown in accordance with various embodiments herein. The carrier layer 204 can include an extension 718. FIG. 8 shows a bottom perspective view of the package 100 shown in FIG. 7. The enclosing layer 208 can include an extension 820. In some embodiments, the carrier layer 204 and the enclosing layer 208 can have the same shape, such as having the same or similar perimeters. In some embodiments, the carrier layer 204 and the enclosing layer 208 can have the same or similar size and shape.

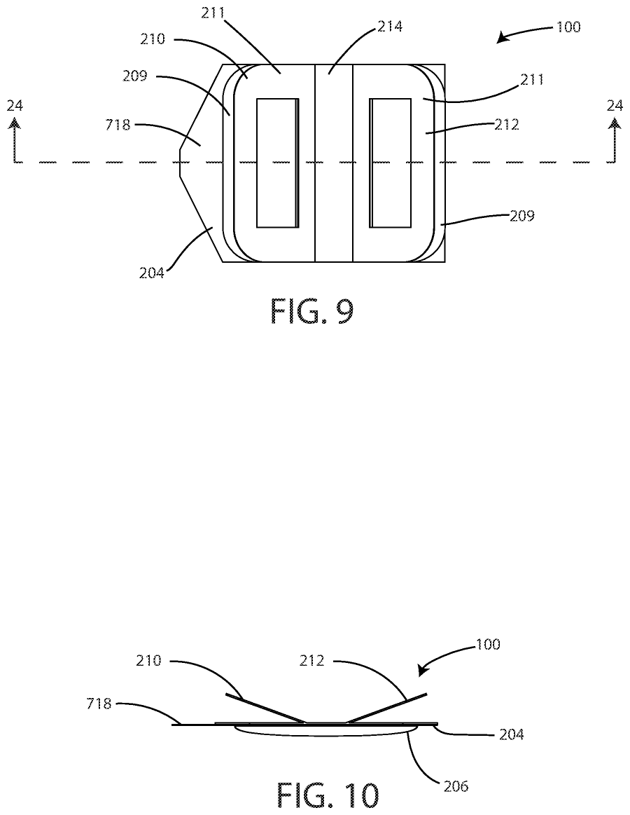

FIG. 9 shows a top view of the package 100 shown in FIG. 7 and FIG. 10 shows a side view of the package 100 shown in FIG. 7. FIGS. 9 and 10 show the package 100 with the extension.

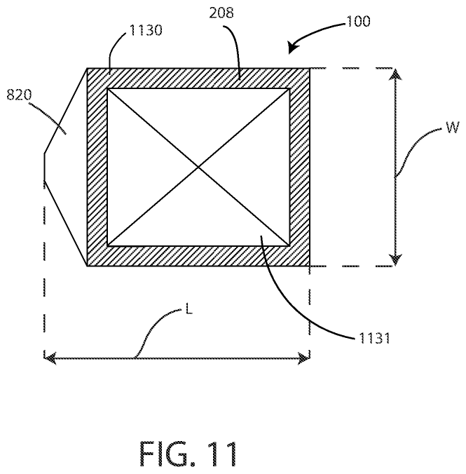

Referring now to FIG. 11, a bottom view of the package 100 shown in FIG. 7 is shown in accordance with various embodiments herein. The bottom view of FIG. 11 shows the enclosing layer 208 with an extension 820 of the package 100. In some embodiments, the enclosing layer 208 can be coupled to the carrier layer 204 by a peelable seal 1130. The area of the peelable seal 1130 is shown in cross-hatching in FIG. 11.

Generally, the peelable seal 1130 is a hermetic bond between the contacting surfaces of the carrier layer 204 and enclosing layer 208. In some embodiments, hermetic can refer to an air tight and liquid tight seal, such that air and liquid cannot pass through the bond. The peelable seal 1130 can be formed by any appropriate method known in the art. In a variety of embodiments, peelable seal 1130 is a heat seal or melt bonded relationship caused by the application of heat and pressure. In an alternate embodiment, peelable seal 1130 is formed with a suitable adhesive applied to at least one of the contacting surfaces of the carrier layer 204 and enclosing layer 208. In at least one embodiment, the peelable seal 1130 is re-sealable, thereby allowing a user to reclose the package 100.

In general, the peelable seal 1130 is configured to have strength conducive to allowing a user to peel the enclosing layer 208 away from and apart from the carrier layer 204 without the use of tools, to access the applicator 206 and the substance 216. In one embodiment, the peelable seal 1130 strength is in the range of 1.25 to 2 pounds per linear inch of seal when pulled apart at 180 degrees at 12 inches per minute travel rate according to testing procedure TAPPI T-494 as published by the Technical Association of the Pulp and Paper Industry. In some embodiments the peelable seal strength is in the range of 1.0 to 2.5 pounds per linear inch of seal. In a variety of embodiments, the peelable seal has strength in the range of 0.25 to 3.5 pounds per linear inch of seal.

In some embodiments, the package 100 can have a length that is about 2.68 inches. In various embodiments, the package 100 can have a length L that is at least 0.5 inches. In various embodiments, the package 100 can have a length L that is at least 0.75 inches. In various embodiments, the package 100 can have a length L that is at least 1 inch. In various embodiments, the package 100 can have a length L that is at least 1.25 inches. In various embodiments, the package 100 can have a length L that is at least 1.5 inches. In various embodiments, the package 100 can have a length L that is at least 2.0 inches. In various embodiments, the package 100 can have a length L that is at least 2.5 inches.

In various embodiments, the package 100 can have a length L that is no more than 10 inches. In various embodiments, the package 100 can have a length L that is no more than 9 inches. In various embodiments, the package 100 can have a length L that is no more than 8 inches. In various embodiments, the package 100 can have a length L that is no more than 7 inches. In various embodiments, the package 100 can have a length L that is no more than 6 inches. In various embodiments, the package 100 can have a length L that is no more than 5 inches. In various embodiments, the package 100 can have a length L that is no more than 4 inches. In various embodiments, the package 100 can have a length L that is no more than 3 inches. In various embodiments, the package 100 can have a length L that is no more than 2.75 inches. In should be understood the package 100 can have a length L that is within a range of any combination of values listed above.

In various embodiments, the package 100 can have a width W of about 2.0 inches. In various embodiments, the package 100 can have a width W of at least 1 inch. In various embodiments, the package 100 can have a width W of at least 1.25 inches. In various embodiments, the package 100 can have a width W of at least 1.5 inches. In various embodiments, the package 100 can have a width W of at least 2.0 inches. In various embodiments, the package 100 can have a width W of at least 2.5 inches. In various embodiments, the package 100 can have a width W of at least 3.0 inches. In various embodiments, the package 100 can have a width W of at least 4.0 inches. In various embodiments, the package 100 can have a width W of at least 5.0 inches.

In various embodiments, the package 100 can have a width W of no more than 8.0 inches. In various embodiments, the package 100 can have a width W of no more than 7.0 inches. In various embodiments, the package 100 can have a width W of no more than 6.0 inches. In various embodiments, the package 100 can have a width W of no more than 5.0 inches. In various embodiments, the package 100 can have a width W of no more than 4.0 inches. In various embodiments, the package 100 can have a width W of no more than 3.0 inches. In various embodiments, the package 100 can have a width W of no more than 2.5 inches. In various embodiments, the package 100 can have a width W of no more than 2.0 inches. In should be understood the package 100 can have a width W that is within a range of any combination of values listed above.

The peelable seal 1130 can surround an unsealed area 1131. The applicator 206 can be disposed in the unsealed area 1131.

In various embodiments, the unsealed area 1131 can have a length of about 1.84 inches. In various embodiments, the unsealed area 1131 can have a length of at least 0.25 inches. In various embodiments, the unsealed area 1131 can have a length of at least 0.5 inches. In various embodiments, the unsealed area 1131 can have a length of at least 0.75 inches. In various embodiments, the unsealed area 1131 can have a length of at least 1.0 inch. In various embodiments, the unsealed area 1131 can have a length of at least 1.25 inches. In various embodiments, the unsealed area 1131 can have a length of at least 1.5 inches. In various embodiments, the unsealed area 1131 can have a length of at least 1.75 inches. In various embodiments, the unsealed area 1131 can have a length of at least 2.0 inches.

In various embodiments, the unsealed area 1131 can have a length of no more than 8.0 inches. In various embodiments, the unsealed area 1131 can have a length of no more than 7.0 inches. In various embodiments, the unsealed area 1131 can have a length of no more than 6.0 inches. In various embodiments, the unsealed area 1131 can have a length of no more than 5.0 inches. In various embodiments, the unsealed area 1131 can have a length of no more than 4.0 inches. In various embodiments, the unsealed area 1131 can have a length of no more than 3.0 inches. In various embodiments, the unsealed area 1131 can have a length of no more than 2.5 inches. In various embodiments, the unsealed area 1131 can have a length of no more than 2.0 inches. In various embodiments, the unsealed area 1131 can have a length of no more than 1.5 inches. In should be understood the unsealed area 1131 can have a length that is within a range of any combination of values listed above.

In various embodiments, the unsealed area 1131 can have a width of about 1.59 inches. In various embodiments, the unsealed area 1131 can have a width of at least 0.5 inches. In various embodiments, the unsealed area 1131 can have a width of at least 0.75 inches. In various embodiments, the unsealed area 1131 can have a width of at least 1.0 inch. In various embodiments, the unsealed area 1131 can have a width of at least 1.5 inches. In various embodiments, the unsealed area 1131 can have a width of at least 2.0 inches.

In various embodiments, the unsealed area 1131 can have a width of no more than 6.0 inches. In various embodiments, the unsealed area 1131 can have a width of no more than 5.0 inches. In various embodiments, the unsealed area 1131 can have a width of no more than 4.0 inches. In various embodiments, the unsealed area 1131 can have a width of no more than 3.0 inches. In various embodiments, the unsealed area 1131 can have a width of no more than 2.0 inches. In various embodiments, the unsealed area 1131 can have a width of no more than 1.75 inches. In various embodiments, the unsealed area 1131 can have a width of no more than 1.5 inches. In should be understood the unsealed area 1131 can have a width that is within a range of any combination of values listed above.

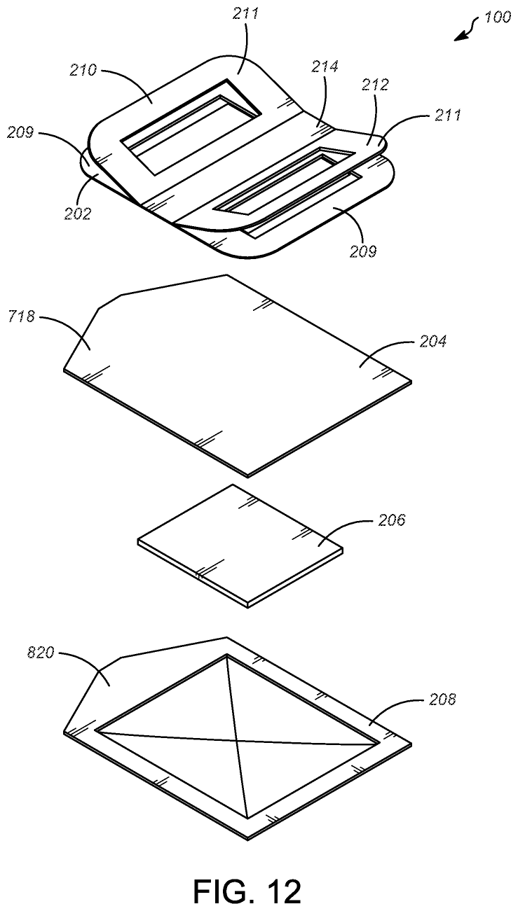

Referring now to FIG. 12, an exploded view of the package 100 shown in FIG. 7 is shown in accordance with various embodiments herein. In some embodiments, the handle layer 202 can be identical to the handle layer 202 described in FIGS. 1-6. The carrier layer 204 can include an extension 718 similar to an extension 820 included in the enclosing layer 208.

### Handle Layer

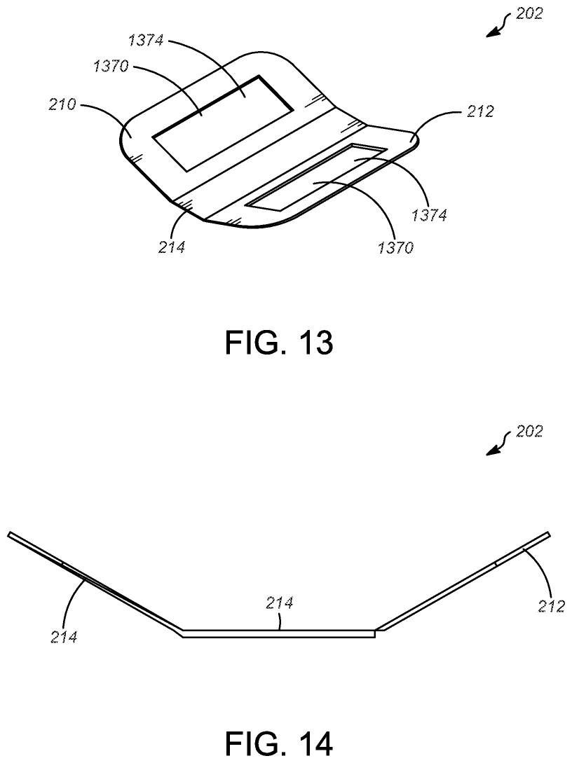

Referring now to FIG. 13, a perspective view of a handle layer 202 is shown in accordance with various embodiments herein. The handle layer 202 includes a first handle portion 210. The handle layer 202 also includes a second handle portion 212. The handle layer 202 also includes a joining portion 214.

In some embodiments, the first handle portion 210 can include a gripping element 1370. Similarly, in some embodiments, the second handle portion 212 can include a gripping element 1370. In various embodiments, the gripping element 1370 includes at least one of an aperture, a projection, and a recess. In the embodiment of FIGS. 13-14, the gripping element 1370 is the aperture 1374 defined by each handle portion. The gripping element 1370 can assist a user in holding the handle layer 202, specifically a handle portion 210, 212, such as while the user transitions the handle portions 210, 212 to the second state from the first state, while the user removes at least a portion of the enclosing layer 208, or while the user applies the substance 216 to a surface.

In some embodiments, the handle layer 202 can include stiff paper, thermoplastic material, polyester, or polypropylene. In various embodiments, the handle layer 202 can be monolithic or one continuous element. In various embodiments, the handle layer 202 has a stiffness of at least 0.1 millinewtons/meter and not more than 1 millinewtons/meter per the Taber stiffness test.

Referring now to FIG. 14, a side view of the handle layer 202 shown in FIG. 13 is shown in accordance with various embodiments herein. The handle layer 202 shown in FIG. 14 includes the first handle portion 210 and the second handle portion 212 in the second state 211.

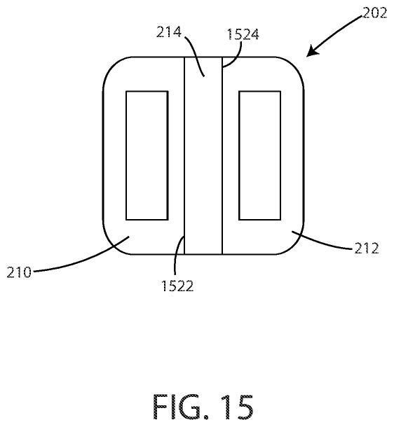

Referring now to FIG. 15, a bottom view of the handle layer 202 shown in FIG. 13 is shown in accordance with various embodiments herein, where the handle portions 210, 212 are bent to be in the second state as illustrated in FIG. 14. In some embodiments, the handle layer 202 includes a first score 1522 and a second score 1524. The first score 1522 and the second score 1524 can each be a recess defined by the handle layer 202, such as a cut or removal of material. The scores 1522, 1524 can define a location where the first handle portion 210 and the second handle portion fold, rotate around, or pivot around. In various embodiments, the first score 1522 can define a boundary between the first handle portion 210 and the joining portion 214. In various embodiments, the second score 1524 can define a boundary between the second handle portion 212 and the joining portion 214. In various embodiments, the first score 1522 and the second score 1524 can extend at least 10% through the thickness of the handle layer 202 and not more than 90% through the thickness of the handle layer 202. In various embodiments, the score is a series of intermittent recesses. In various embodiments, the score is a continuous recess. In some embodiments, the first score 1522 and the second score 1524 can extend all the way across the width of the handle layer 202, such as shown in FIG. 15, from a first side to a second side.

### Carrier Layer

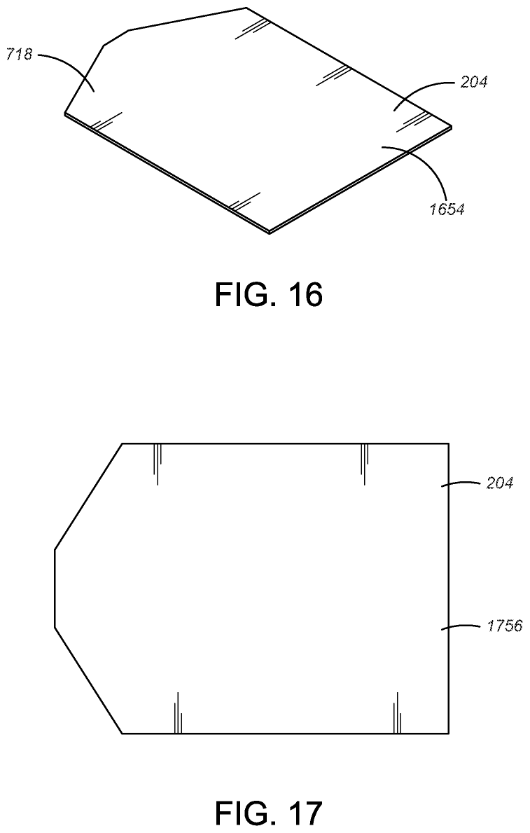

Referring now to FIGS. 16 and 17, a perspective view of the carrier layer 204 is shown in FIG. 16 and a bottom view of the carrier layer 204 is shown in FIG. 17, both in accordance with various embodiments herein. The carrier layer 204 can include a first surface 1654 and a second surface 1756. The first surface 1654 can be opposite from the second surface 1756. In various embodiments, the first surface 1654 and the second surface 1756 can be flat or planar.

In various embodiments, the carrier layer 204 provides a barrier for moisture, air and/or light. In various embodiments, the carrier layer 204 has a moisture vapor transmission rate (MVTR) of at least 0.0005 g/100 in² per 24 hours at 90% relative humidity and 40° C. and not more than 5 g/100 in² per 24 hours at 90% relative humidity and 40° C. per ASTM F1245. In various embodiments, the carrier layer 204 has an oxygen transmission rate (OTR) of at least 0.0005 cc/100 in² per 24 hours at 0% relative humidity and 23° C. and not more than 500 cc/100 in² per 24 hours at 0% relative humidity and 23° C. per ASTM D3985.

In various embodiments, the carrier layer 204 can include a multilayer laminate, such as a foil polymer paper laminate. In various embodiments, the various layers in a multilayer laminate can provide different properties, such as one layer for a moisture barrier and a second layer for structural integrity.

### Applicator

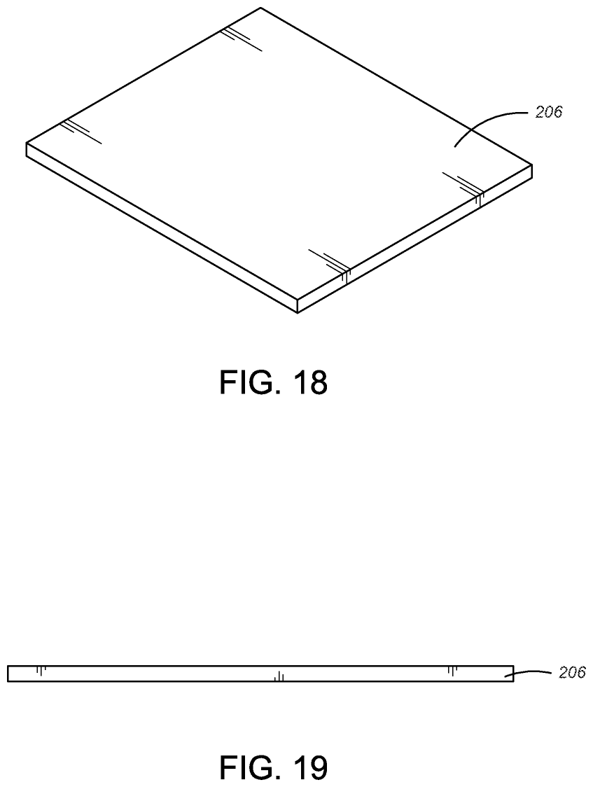

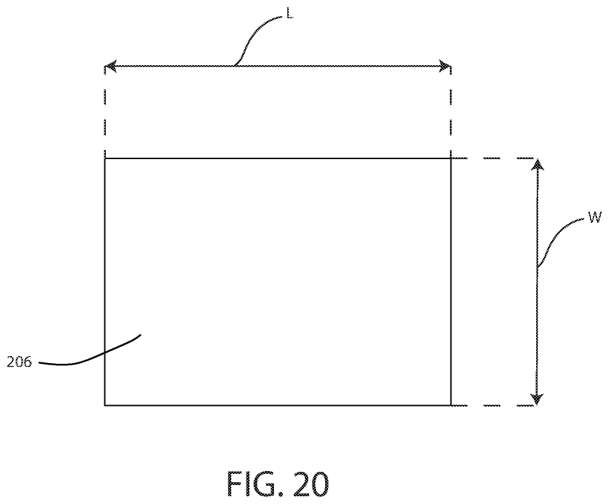

Referring now to FIGS. 18-20, a perspective view of the applicator 206 is shown in FIG. 18, a side view of the applicator 206 is shown in FIG. 19, and a top view of the applicator 206 is shown in FIG. 20, all in accordance with various embodiments herein. In various embodiments, the applicator 206 can include at least one flat or planar surface, or at least two flat or planar surfaces. In some embodiments, the two flat or planar surfaces are opposite from each other. In some embodiments, the applicator 206 is substantially a rectangular prism.

In some embodiments, the applicator 206 holds the substance 216 in pores of the applicator 206. In various embodiments, the applicator 206 can include an absorbent material, such as cloth, paper, porous polymer, foam, or sponge.

In other embodiments, the applicator can include a non-absorbent material, such as a closed cell foam. In such embodiments, the applicator 206 can hold the substance 216 on a surface of the applicator 206.

In various embodiments, the applicator 206 has a stiffness of at least 0 millinewtons/meter and not more than 1 millinewtons/meter per the Taber stiffness test.

In various embodiments, the applicator 206 can have a length L of about 1.5 inches. In various embodiments, the applicator 206 can have a length L of at least 1.0 inch. In various embodiments, the applicator 206 can have a length L of at least 1.25 inches. In various embodiments, the applicator 206 can have a length L of at least 1.5 inches. In various embodiments, the applicator 206 can have a length L of at least 1.75 inches. In various embodiments, the applicator 206 can have a length L of at least 2.0 inches. In various embodiments, the applicator 206 can have a length L of at least 3.0 inches. In various embodiments, the applicator 206 can have a length L of at least 4.0 inches. In various embodiments, the applicator 206 can have a length L of at least 5.0 inches.

In various embodiments, the applicator 206 can have a length L of no more than 6.0 inches. In various embodiments, the applicator 206 can have a length L of no more than 5.0 inches. In various embodiments, the applicator 206 can have a length L of no more than 4.0 inches. In various embodiments, the applicator 206 can have a length L of no more than 3.0 inches. In various embodiments, the applicator 206 can have a length L of no more than 2.0 inches. In should be understood the applicator 206 can have a length L that is within a range of any combination of values listed above.

In various embodiments, the applicator 206 can have a width W of about 1.25 inches. In various embodiments, the applicator 206 can have a width W of at least 1.0 inch. In various embodiments, the applicator 206 can have a width W of at least 1.25 inches. In various embodiments, the applicator 206 can have a width W of at least 1.5 inches. In various embodiments, the applicator 206 can have a width W of at least 2.0 inches. In various embodiments, the applicator 206 can have a width W of at least 3.0 inches. In various embodiments, the applicator 206 can have a width W of at least 4.0 inches.

In various embodiments, the applicator 206 can have a width W of no more than 5.0 inches. In various embodiments, the applicator 206 can have a width W of no more than 4.0 inches. In various embodiments, the applicator 206 can have a width W of no more than 3.0 inches. In various embodiments, the applicator 206 can have a width W of no more than 2.0 inches. In various embodiments, the applicator 206 can have a width W of no more than 1.5 inches. In various embodiments, the applicator 206 can have a width W of no more than 1.25 inches. In various embodiments, the applicator 206 can have a width W of no more than 1.0 inch. In should be understood the applicator 206 can have a width W that is within a range of any combination of values listed above.

### Substance

In various embodiments, the substance on or held by the applicator can be at least partially solid or at least partially liquid. In some embodiments, the substance can be entirely a solid or entirely a liquid. In some embodiments, the substance can be at least partially solid and at least partially liquid. In various embodiments, the substance can include a cream or a gel.

In some embodiments, the substance can include medicated gel or cream. In some embodiments, the substance can include a paint, a stain or a dye.

### Enclosing Layer

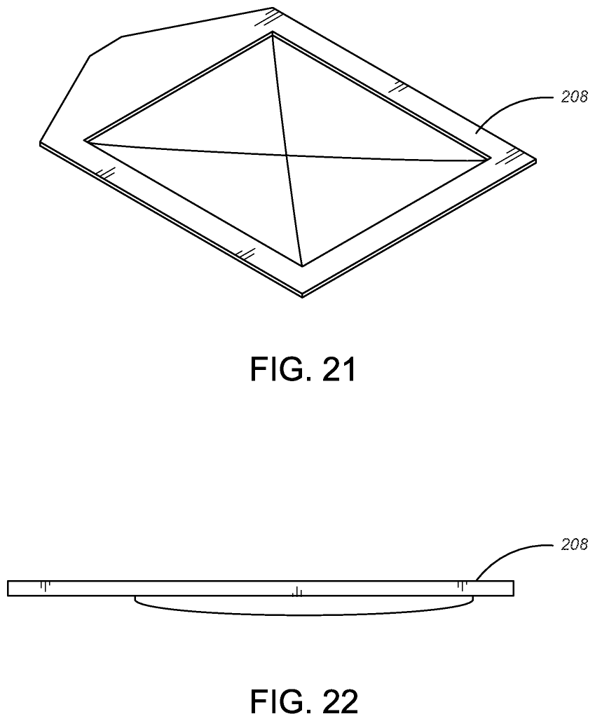

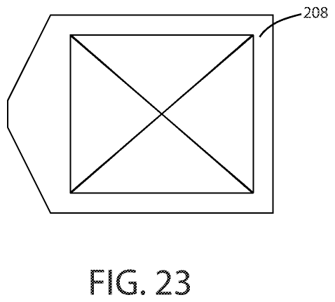

Referring now to FIGS. 21-23, a perspective view of the enclosing layer 208 is shown in FIG. 21, a side view of the enclosing layer 208 is shown in FIG. 22, and a top view of the enclosing layer 208 is shown in FIG. 23, all in accordance with various embodiments herein. In some embodiments, the enclosing layer 208 can be non-planar, such as to define a cavity or recess to accommodate the applicator 206, such as best shown in FIG. 22. The shape of the enclosing layer is also indicated by the crossed lines showing curvature toward the viewer in FIG. 23. In other embodiments, the enclosing layer 208 can include two flat or planar surfaces, similar to the carrier layer 204.

In various embodiments, the enclosing layer 208 provides a barrier for moisture, air and/or light. In various embodiments, the enclosing layer 208 has a moisture vapor transmission rate (MVTR) of at least 0.0005 g/100 in² per 24 hours at 90% relative humidity and 40° C. and not more than 5 g/100 in² per 24 hours at 90% relative humidity and 40° C. per ASTM F1245. In various embodiments, the enclosing layer 208 has an oxygen transmission rate (OTR) of at least 0.0005 cc/100 in² per 24 hours at 0% relative humidity and 23° C. and not more than 500 cc/100 in² per 24 hours at 0% relative humidity and 23° C. per ASTM D3985.

### Steps for Applying the Substance

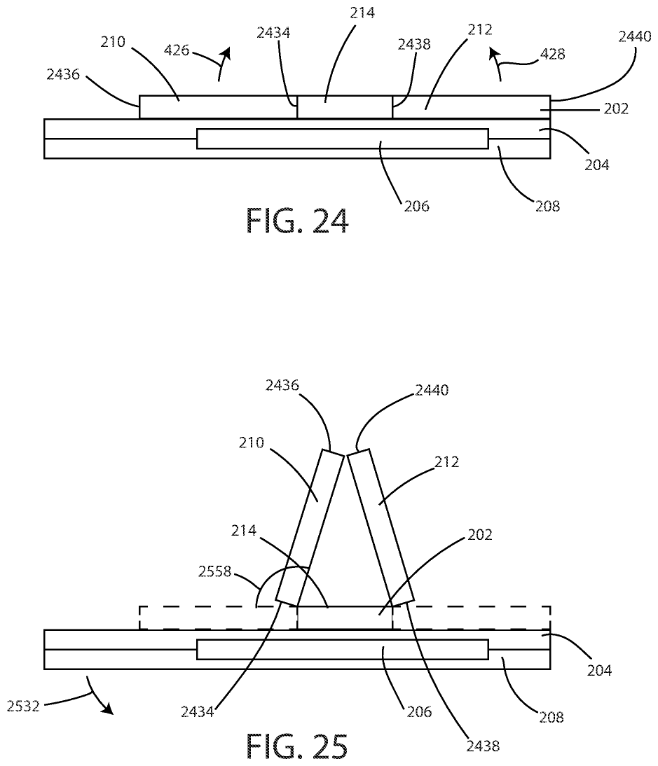

FIGS. 24-26 are each side, cross-sectional, schematic views of a package and show various steps in a sequence of exposing the substance in preparation for applying or using the substance. The cross-section of FIGS. 24-26 is taken through a longitudinal center line as illustrated by line 24-24 of FIG. 9. FIGS. 24-26 show some components in a manner that is exaggerated and not to scale in order to better illustrate the components and their relationships.

Referring now to FIG. 24, a package 100 is shown in accordance with various embodiments herein. The package 100 can include a handle layer 202. The package 100 can also include a carrier layer 204 and an enclosing layer 208. The package 100 can further include an applicator 206 disposed between the carrier layer and the enclosing layer 208.

The handle layer 202 can include a first handle portion 210 and a second handle portion 212 joined by a joining portion 214. The first handle portion 210 can include a first end 2434 and a second end 2436. The second handle portion 212 can include a first end 2438 and a second end 2440.

In various embodiments, the first end 2434 of the first handle portion 210 is adjacent to the joining portion 214 and the first end 2438 of the second handle portion 212 is adjacent to the joining portion 214. In various embodiments, in the first state 209, the first handle portion 210 can be coplanar with the second handle portion 212.

Referring now to FIG. 25, the package 100 is shown with the first handle portion 210 and the second handle portion 212 transitioned into the second state 211 in accordance with various embodiments herein. The dashed lines in FIG. 25 can represent the position of the first handle portion 210 and the second handle portion 212 in the first state.

In some embodiments, the angle 2558 between the first state 209 and the second state 211 can be at least 15° and not more than 130°. In various embodiments, the angle 2558 between the first state 209 and the second state 211 for the first handle portion 210 can have the same range as a similar angle for the second handle portion 212.

In various embodiments, in the second state 211, the first handle portion 210 and the second handle portion 212 are substantially perpendicular with the carrier layer 204. In various embodiment, in the second state, the angle 2558 between the first state 209 and the second state 211 can be greater than 90 degrees so that the first handle portion 210 and second handle portion 212 can contact each other. The angle 2558 is influenced by the length of the joining portion 214. A longer joining portion 214 will result in a larger angle 2558 when the first handle portion 210 is contacting the second handle portion 212. In one embodiment, the angle 2558 between the first state 209 and the second state 211 can be about 120 degrees.

In various embodiments, in the second state 211, the first handle portion 210 can contact the second handle portion 212, such as the second end 2436 contacting the second end 2440. In this configuration, the user can squeeze the second end 2436 against the second end 2440 when grasping the handle portions. For example, the user may squeeze the second end 2436 against the second end 2440 using a thumb and forefinger or thumb and middle finger.

To expose the applicator 206 and substance 216, the enclosing layer 208 can be removed from the package 100, such as by pulling or moving the enclosing layer 208 away from the remainder of the package 100 shown by arrow 2532.

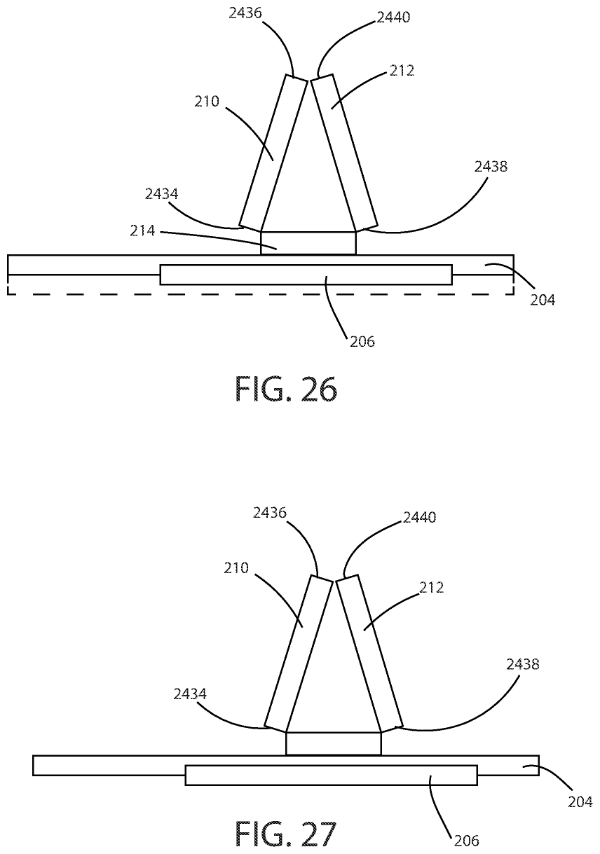

Referring now to FIG. 26, the package 100 is shown with the enclosing layer 208 removed. The previous location of the enclosing layer 208 is shown in dashed lines. FIG. 27 shows a schematic of the package 100 with the enclosing layer 208 removed and ready for the substance 216 on the applicator 206 to be applied to a desired location.

In various embodiments, in the first state 209, the second end 2436 of the first handle portion 210 and the second end 2436 the second handle portion 212 are further apart than when the first handle portion 210 and the second handle portion 212 are in the second state 211, such as shown when comparing FIG. 24 with FIG. 27. Similarly, the second end 2436 and the second end 2440 can be closer in the second state 211 than in the first state 209.

### Partially Removeable Enclosing Layer

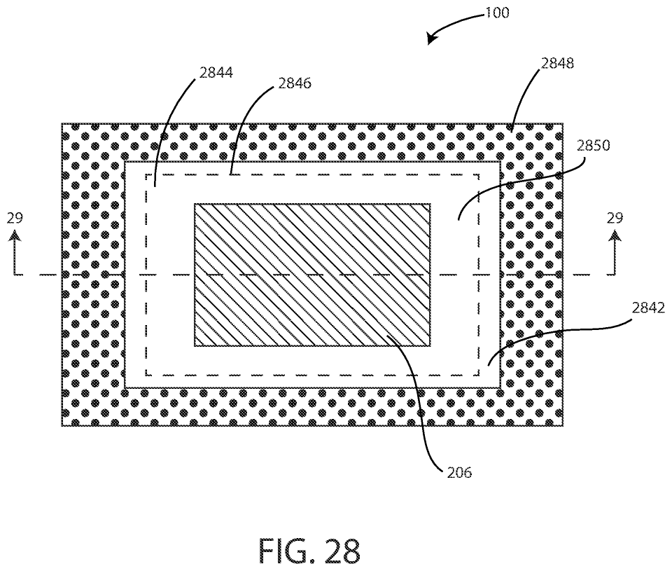

Referring now to FIG. 28, a bottom schematic view of a package 100 is shown in accordance with various embodiments herein. As discussed above, the package 100 can include an enclosing layer coupled to the carrier layer. In some embodiments, the coupling can include a peelable seal, such as shown in FIG. 11.

In various embodiments, a portion of the enclosing layer 208 is removeable, such as the removeable portion 2844. In various embodiments, a portion of the enclosing layer 208 is fixed to the carrier layer, such as the fixed portion 2848, and a portion of the enclosing layer 208 is not fixed to the carrier layer, such as the unfixed portion 2850.

In some embodiments, the coupling between the carrier layer and the enclosing layer can include a fixed portion 2848. The fixed portion 2848 can include a coupling between the carrier layer and the enclosing layer that is permanent, such that the layers themselves would be destroyed before the coupling. For example, the fixed portion 2848 can include the enclosing layer welded to the carrier layer or fixed via an adhesive. The package 100 can further include an unfixed portion 2850. The unfixed portion 2850 can be a portion of the package 100 where the carrier layer is not coupled to the enclosing layer, such that an air gap or the applicator 206 can be disposed between the enclosing layer and the carrier layer. In various embodiments, the unfixed portion 2850 can also include portions of the carrier layer contacting, but not coupled or attached to, the enclosing layer.

In some embodiments, the enclosing layer can include micro-perforations 2846, such as a series of laser cut recesses in the enclosing layer. The micro-perforations 2846 can surround the removeable portion 2844. The micro-perforations 2846 can define an enclosed area. The portion of the enclosing layer that is within the enclosed area can be removed, such as to expose the applicator 206 and the substance 216.

The enclosing layer 208 can include a removeable portion 2844 and a remaining portion 2842. The removeable portion 2844 can be separated from the remaining portion 2842 and the rest of the package 100 to expose the applicator 206. The boundary between the removeable portion 2844 and the remaining portion 2842 can be defined by the micro-perforations 2846. In various embodiments, the remaining portion 2842 can include a portion of the unfixed portion 2850 and the fixed portion 2848, such as when the micro-perforations 2846 extend through the unfixed portion 2850. The removeable portion 2844 can be surrounded by the micro-perforations 2846.

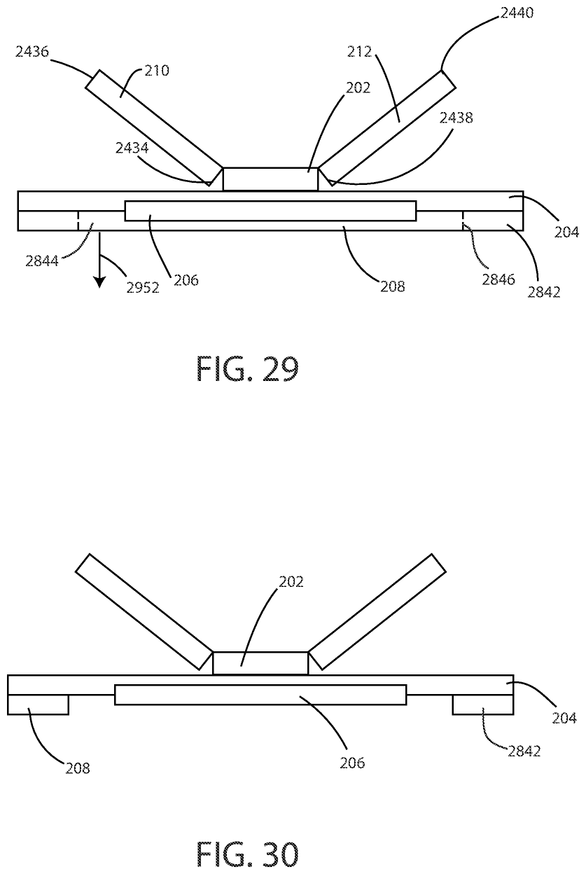

Referring now to FIGS. 29-30, a cross-sectional side view of a package 100 is shown, where the cross-section is taken through a longitudinal center line as illustrated by line 29-29 of FIG. 28. FIGS. 29-30 show some components in a manner that is exaggerated and not to scale in order to better illustrate the components and their relationships. In FIG. 29 the package 100 is shown in accordance with various embodiments herein. The enclosing layer 208 can include a remaining portion 2842 and a removeable portion 2844. Micro-perforations 2846 can define a boundary between the portion that can be removed and the portion that will remain attached to the carrier layer 204. It should be understood that the handle portions 210, 212 can be transitioned into the second state in the same manner as discussed above and shown in FIGS. 24-25.

The removeable portion 2844 can be removed or separated from the package 100, such as by pulling, peeling, or otherwise removing the removeable portion 2844 away from the remaining portion 2842 and the carrier layer 204 in the direction of arrow 2952. In various embodiments, the user will start the peeling process from one of the shorter ends of the removeable portion 2844.

Referring now to FIG. 30, the package 100 shown in FIG. 29 is shown with the removeable portion having been removed and the applicator 206 being exposed. A frame portion of the enclosing layer 208 is left behind after the removeable portion 2844 is gone.

### Package Options

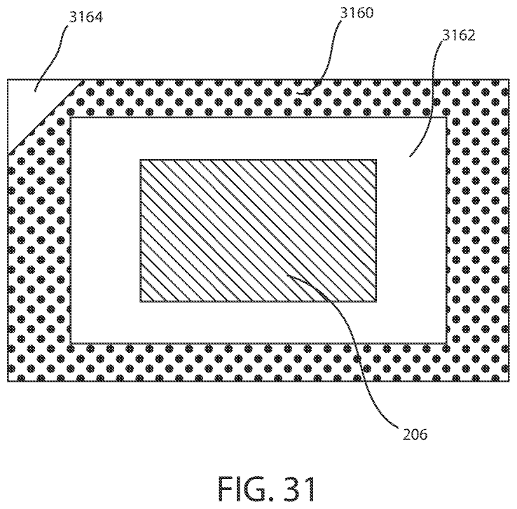

Referring now to FIG. 31, a bottom schematic view of a package 100 is shown in accordance with various embodiments herein. In various embodiments, the enclosing layer can be coupled to the carrier layer via a peelable seal 3160. The peelable seal 3160 can surround an unsealed area 3162. The applicator 206 can be disposed within the unsealed area 3162. In various embodiments, the package 100 can include a tab 3164. The tab 3164 can be an unsealed portion of the enclosing layer and the carrier layer that is outside of the peelable seal, such that the tab is separated from the unsealed area 3162 that contains the applicator 206 by the peelable seal 3160. The tab 3164 can be an unfixed portion of the enclosing layer and carrier layer that is external to the unsealed area 3162. The tab 3164 can aid a user in removing the enclosing layer from the package 100, such as by providing a location to start peeling the enclosing layer from the package 100.

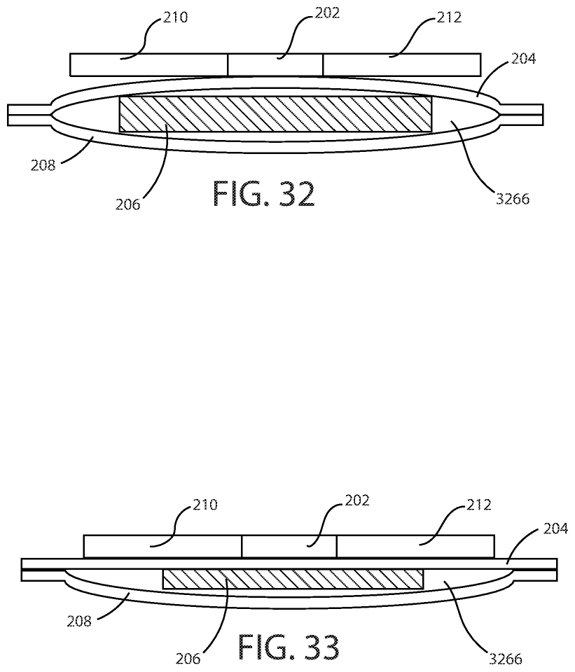

Referring now to FIG. 32, a side, cross-sectional view of a package 100 is shown, where the cross-section is taken through a longitudinal center line, in accordance with various embodiments herein. In some embodiments, the carrier layer 204 and the enclosing layer 208 can both be non-planar, such as to form a cavity 3266 to hold an applicator 206. The carrier layer 204 and/or the enclosing layer 208 can be non-planar, such that the layers are non-planar even without an applicator disposed between. The carrier layer and/or enclosing layer can be non-planar by themselves, such as before assembling the package 100. A non-planar layer can have more surface area than a planar layer that extends between the same two points. In various embodiments, the carrier layer 204 and the enclosing layer 208 can have the same surface area.

Referring now to FIG. 33, a side, cross-sectional view of a package 100 is shown, where the cross-section is taken through a longitudinal center line, in accordance with various embodiments herein. In some embodiments, the carrier layer 204 can be planar and the enclosing layer 208 can be non-planar, such as shown in FIG. 33. In some embodiments, the enclosing layer 208 can have a larger surface area than the carrier layer.

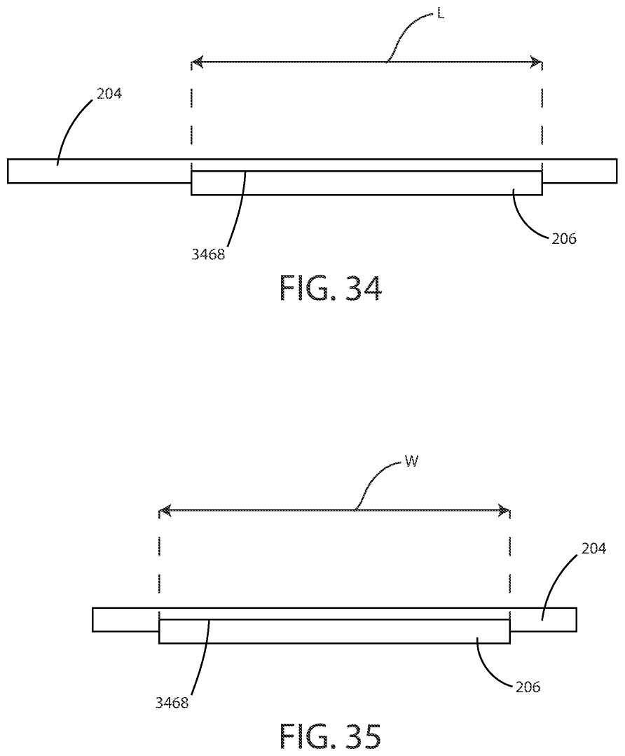

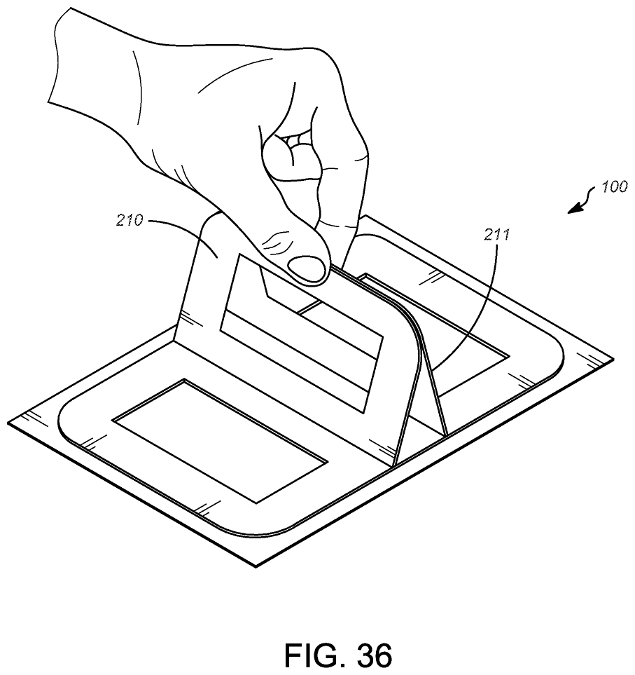

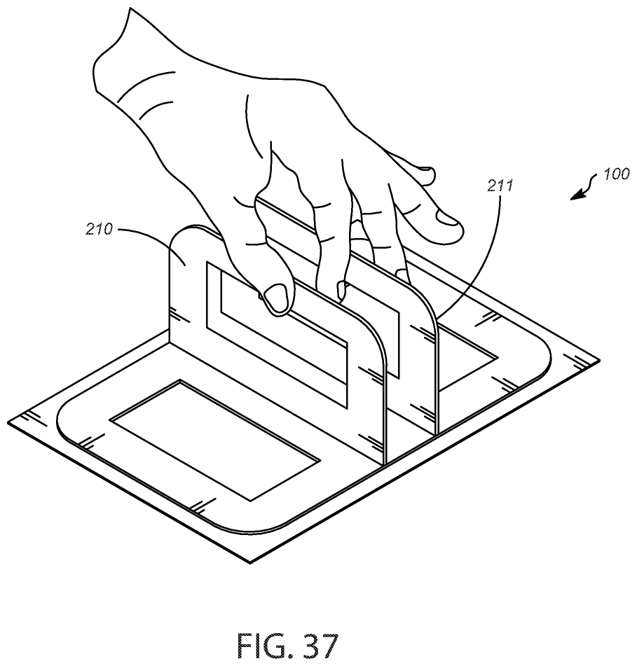

Referring now to FIGS. 34 and 35, the applicator 206 can be coupled to the carrier layer 204 via a compression fit. In various embodiments, the carrier layer 204 can define a recess 3468 that is configured to receive and hold the applicator 206.

FIG. 34 is a side, cross-sectional view of the package 100 taken through a longitudinal center line, in accordance with various embodiments herein, so that a length L of the applicator can be illustrated. FIG. 35 is an end, cross-sectional view of the package 100 taken through a lateral center line, in accordance with various embodiments herein, so that a width W of the applicator can be illustrated In some embodiments, the applicator 206 can have a slightly larger length L than the recess 3468, such that the applicator 206 is compressed within the recess 3468. Similarly, in some embodiments, the applicator 206 can have a slightly larger width W than the recess 3468, such that the applicator 206 is compressed within the recess 3468. In some embodiments, the recess 3468 can be coated with an adhesive to aid in retaining the applicator 206 in the recess 3468.

### Holding of Package

FIGS. 36 and 37 show two potential ways in which the package 100 can be used to apply the substance. FIG. 36 shows an embodiment of the package where the user holds the two handle portions 210, 212 together to apply the substance. FIG. 37 shows an embodiment of the package where the user inserts one or more of his/her fingers between the two handle portions 210, 212 to apply the substance. In some embodiments, a package 100 can be used in either manner depending on the user's needs or the user's preferences.

In reference to FIG. 36, a user's hand is shown holding the first handle portion 210 and the second handle portion 212 to apply the substance. The first handle portion 210 and the second handle portion 212 are in the second state 211. A user can grasp the two handle portions 210, 212 between two of his/her fingers, such as a thumb and a first finger (index finger). The two handle portions 210, 212 can contact each other as the user holds them together. While holding the two handle portions 210, 212 the user can apply the substance to a desired surface, while avoiding direct contact between the substance and the user's hand.

In reference to FIG. 37, a user's hand is shown holding the first handle portion 210 and the second handle portion 212 with one of the user's fingers separating the handle portions 210, 212. In some embodiments, the user can have a finger, such as the user's first finger, disposed between the two handle portions 210, 212, while the user's thumb and second finger (middle finger) press the two handle portions 210, 212 together. In some instances (not shown in FIG. 37), the user's first finger can apply pressure to the top of the handle layer 202 at the joining portion 214, such as to apply additional pressure while applying the substance.

### Manufacturing

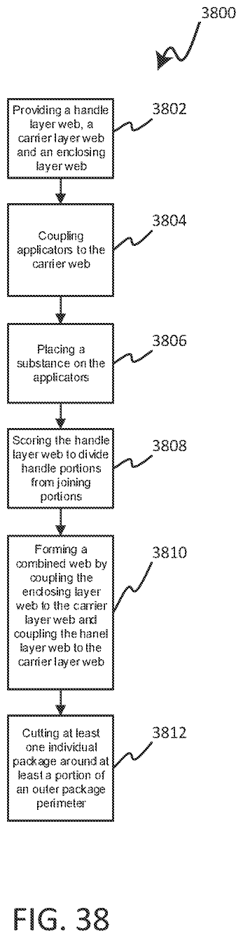

FIG. 38 depicts a flow chart consistent with a method of manufacturing a package in accordance with various embodiments herein. Generally, applicators can be coupled to a carrier layer web. The substance can be added to the applicators before or after the applicators are disposed on to the carrier layer web.

In embodiments using micro-perforations in the enclosing layer, the enclosing layer web can go through a process to add the microperforated boundary between the removable portion and the remaining portion before the enclosing layer is coupled to the carrier layer. An enclosing layer web can be applied to the carrier layer web with the applicators located between the enclosing layer web and the carrier layer web. The carrier layer web and the enclosing layer web can be coupled together at a seal area.

A handle layer can go through a die-cut process to create the aperture portions and create the scores between the joining portion and the first and second handle portions. The die-cut process may happen before the handle layer is joined to the carrier layer or after the handle layer is joined to the carrier layer.

A handle layer can be coupled to the opposite side of the carrier layer web from the applicator and enclosing layer web before or after the coupling with the applicator and/or the coupling with the enclosing layer. The webs can be cut to separate the individual packages

Many different methods are contemplated herein, including, but not limited to, methods of making, methods of using, and the like. Aspects of system/device operation described elsewhere herein can be performed as operations of one or more methods in accordance with various embodiments herein.

In an embodiment, a method 3800 of making a package, is included, the method 3800 can include providing a handle layer web, a carrier layer web, and an enclosing layer web 3802. The method 3800 can include coupling applicators to the carrier layer web 3804. The method 3800 can include placing a substance on the applicators 3806. In some embodiments, placing a substance on the applicators occurs after the applicators are coupled to the carrier layer web. In some embodiments, placing a substance on the applicators occurs before the applicators are coupled to the carrier layer web. The method 3800 can further include scoring the handle layer web to divide handle portions from joining portions 3808. In some embodiments, the method 3800 can include forming a combined web by coupling the enclosing layer web to the carrier layer web and coupling the handle layer web to the carrier layer web 3810. In some embodiments, forming the combined web can enclose the applicators between the carrier layer web and the enclosing layer. In various embodiments, the method 3800 can include cutting at least one individual package around at least a portion of an outer package perimeter to separate the individual package from the combined web 3812. In various embodiments, a first handle portion and a second handle portion of the individual package are configured to move from a first state to a second state, wherein, in the first state, the first handle portion and the second handle portion are parallel with the carrier layer, and, in the second state, the first handle portion and the second handle portion are not parallel to the carrier layer. In an embodiment of the method 3800, cutting the individual package comprises die cutting or rotary cutting.

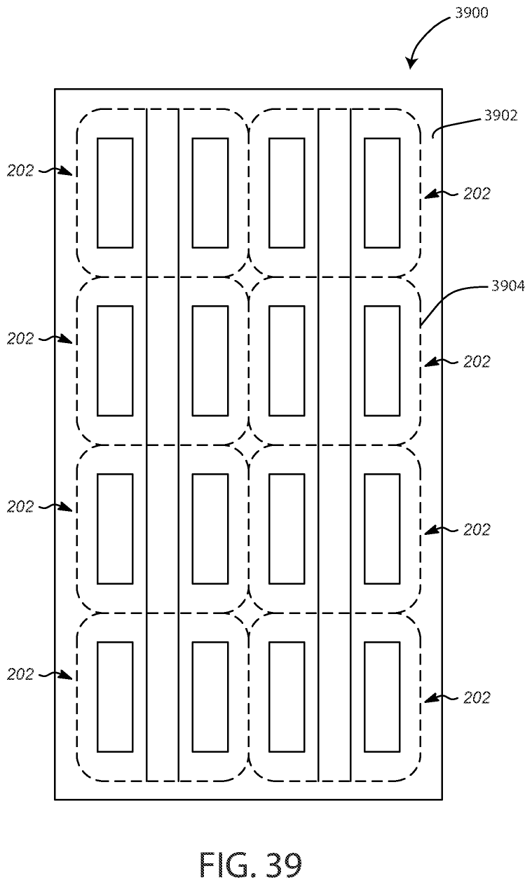

FIG. 39 shows a top view of a handle layer web 3900 in accordance with various embodiments. It can be seen in FIG. 39 that the handle layer web 3900 can include a plurality of handle layers 202 for a plurality of packages. In some embodiments, there can be a web 3902 that can hold the plurality of handle layers 202 together. To separate the individual handle layers 202 from each other and from the web 3902, the handle layer web 3900 can be cut along the dashed lines 3904. It should be understood that the other layers can be manufactured in a similar manner as the handle layer shown in FIG. 39. It is also considered for a plurality of packages to be assembled on one of the layer webs. After the packages are assembled on one web, the packages can be cut or otherwise separated from each other and from the web.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A package for containing and applying a substance, the package comprising:
a handle layer comprising:
a first handle portion;
a second handle portion; and
a joining portion between the first handle portion and the second handle portion;
a carrier layer comprising:
a first surface; and
a second surface opposite from the first surface;
wherein the joining portion of the handle layer is connected to the first surface of the carrier layer;
an applicator containing the substance; and
an enclosing layer coupled to the second surface of the carrier layer, wherein the enclosing layer, and the carrier layer define a cavity with the applicator disposed in the cavity, such that the applicator is disposed between the carrier layer and the enclosing layer;
wherein the first handle portion and the second handle portion are configured to move from a first state to a second state, wherein, in the first state, the first handle portion and the second handle portion are parallel with the carrier layer, and, in the second state, the first handle portion and the second handle portion are not parallel to the carrier layer.

2. The package of claim 1, wherein the applicator holds the substance in pores of the applicator.

3. The package of claim 1, wherein the applicator comprises an absorbent material, cloth, paper, porous polymer, foam, or sponge.

4. The package of claim 1, wherein the applicator is bonded to the second surface of the carrier layer.

5. The package of claim 1, wherein a first score defines a boundary between the first handle portion and the joining portion, and a second score defines a boundary between the second handle portion and the joining portion.

6. The package of claim 5, wherein the first score and the second score extend at least 10% through the handle layer and not more than 90% through the handle layer.

7. The package of claim 1, wherein the first handle portion and the second handle portion rotate in opposite directions to move between the first state to the second state.

8. The package of claim 1, wherein in the first state, the first handle portion is coplanar with the second handle portion.

9. The package of claim 1, wherein the first handle portion and the second handle portion each include a gripping element.

10. The package of claim 9, wherein the gripping element includes at least one of an aperture, a projection, and a recess.

11. The package of claim 1, wherein the handle layer comprises stiff paper, thermoplastic material, polyester, or polypropylene.

12. The package of claim 1, wherein a first angle between the first handle portion in the first state and the first handle portion in the second state is at least 15° and not more than 130°, and a second angle between the second handle portion in the first state and the second handle portion in the second state is at least 15° and not more than 130°.

13. The package of claim 1, wherein the carrier layer comprises a foil polymer paper laminate.

14. The package of claim 1, wherein a portion of the enclosing layer is removeable.

15. The package of claim 14, wherein a portion of the enclosing layer is fixed to the package, and a portion of the enclosing layer is not fixed to the package.

16. The package of claim 15, wherein micro-perforations define a boundary between the portion of the enclosing layer that is removeable and a portion of the enclosing layer which is not removeable.

17. The package of claim 1, wherein the enclosing layer is coupled to the carrier layer with a peelable seal.

18. A package for containing and applying a substance, the package comprising:
a handle layer comprising:
a first handle portion comprising a first end and a second end;
a second handle portion comprising a first end and a second end; and
a joining portion between the first handle portion and the second handle portion, wherein the first end of the first handle portion is adjacent to the joining portion and the first end of the second handle portion is adjacent to the joining portion;
a carrier layer comprising:
a first surface; and
a second surface opposite from the first surface;
wherein the joining portion of the handle layer is connected to the first surface of the carrier layer;
an applicator containing the substance; and
an enclosing layer coupled to the carrier layer, wherein the enclosing layer, and the carrier layer define a cavity with the applicator disposed in the cavity, such that the applicator is disposed between the carrier layer and the enclosing layer;
wherein the first handle portion and the second handle portion are configured to move from a first state to a second state, wherein in the first state the second end of the first handle portion and the second end the second handle portion are further apart than when the first handle portion and the second handle portion are in the second state.

19. A method of making a packing, comprising:
providing a handle layer web, a carrier layer web, and an enclosing layer web;
coupling applicators to the carrier layer web;
placing a substance on the applicators;
scoring the handle layer web to divide handle portions from joining portions;
forming a combined web by coupling the enclosing layer web to the carrier layer web to enclose the applicators and coupling the handle layer web to the carrier layer web; and
cutting at least one individual package around at least a portion of an outer package perimeter to separate the individual package from the combined web;
wherein a first handle portion and a second handle portion of the individual package are configured to move from a first state to a second state, wherein, in the first state, the first handle portion and the second handle portion are parallel with the carrier layer, and, in the second state, the first handle portion and the second handle portion are not parallel to the carrier layer.

20. The method of claim 19, wherein cutting the individual package comprises die cutting or rotary cutting.
